(19) United States
(12) Patent Application Publication
(10) Pub. No.: US 2026/0128177 A1
(43) Pub. Date: May 7, 2026

(54) SYSTEMS AND METHODS FOR PROCESSING OF FUNDUS IMAGES

(71) Applicant: TOKU EYES LIMITED, Newmarket (NZ)

(72) Inventors: Seyed Ehsan Vaghefi Rezaei, San Diego, CA (US); David Michael Squirrell, Sunshine Coast (AU); Song Yang, Auckland (NZ); Songyang An, Auckland (NZ); Li Xie, Auckland (NZ); Michael Vincent Carroll McConnell, San Francisco, CA (US); Shima Mohammadi Moghadam, Auckland (NZ)

(21) Appl. No.: 19/377,742

(22) Filed: Nov. 3, 2025

Related U.S. Application Data
(60) Provisional application No. 63/715,370, filed on Nov. 1, 2024.

(51) Int. Cl. G16H 50/30; A61B 3/12; A61B 5/00; A61B 5/20; G16H 50/20

(52) U.S. Cl. CPC G16H 50/30; A61B 3/12; A61B 5/201; A61B 5/7267; A61B 5/7275; G16H 50/20

(57) ABSTRACT

Systems and methods for determining one or more recommendations for management of wellbeing of an individual are disclosed. An indication of risk of chronic kidney disease (CKD) is determined by a deep learning model based on one or more fundus images. Recommendations for management of the individual's wellbeing are based at least in part on the determined indication of risk of CKD.

### CROSS-REFERENCE TO RELATED APPLICATION

This application claims priority to U.S. patent application No. 63/715,370, filed Nov. 1, 2024, the entire contents of which are incorporated herein by reference.

### TECHNICAL FIELD

The present technology relates to systems and methods for processing fundus images, more particularly the processing of fundus images to determine a risk of chronic kidney disease.

### BACKGROUND

Chronic kidney disease (CKD) is well recognized as a major life-threatening and debilitating disease. CKD, when left untreated, has a high risk of progressing to kidney failure requiring dialysis or kidney transplantation. In addition, CKD can be an indicator of and contributor to multiple cardiovascular and metabolic diseases, including type 2 diabetes (DM), hypertension (HTN), atherosclerotic cardiovascular disease (ASCVD), heart failure (HF), and arrhythmia (both atrial fibrillation and sudden death). The critical role played by CKD in the development of multiple chronic health conditions is exemplified by the cardiovascular-kidney-metabolic syndrome, as recently highlighted by the American Heart Association. Impaired kidney function is increasingly recognized as a key mediator of the relations between metabolic risk factors, such as obesity and DM, and cardiovascular disease, including heart failure (HF). DM and HTN can lead to CKD, and CKD can lead to HTN. The role of CKD in patients with DM is particularly profound. Per data from the National Health and Nutrition Examination Survey (NHANES) linked to the National Death Index, CKD was present in 42% of patients with DM. Furthermore, their mortality was substantially increased when CKD also present, with the excess mortality reaching 47% in diabetic individuals with both albuminuria and impaired GFR.

Kidney disease is defined as an abnormality of kidney structure or function, which can resolve or become chronic. CKD is a general term for disorders affecting kidney structure and function with variable severity, clinical presentation, and rate of progression. CKD affects about 15% of the U.S. population, but 9 out of 10 people with CKD are unaware that they have the condition. CKD is more prevalent in Black persons and women and among persons 60 years and older, with more advanced disease associated with an increased risk of cardiovascular disease and death. Earlier stages of CKD are typically asymptomatic and progression to more advanced stages can be prevented or delayed. Progression of CKD can lead to kidney failure, which often requires treatment by dialysis or transplantation. Dialysis and/or kidney transplantation are among the most costly of chronic diseases (in terms of both burden and expense) and significantly reduce lifespan. Their costs consume a disproportionate amount of healthcare budgets. The Medicare program spends more than $130 billion-over 24 percent of total spending-on patients with kidney disease. Treatment for kidney failure and its complications represents approximately 7% of the Medicare budget, for less than 0.1% of the population. Failure to recognize CKD results in more severe complications, and late referral, results in worse outcomes even with therapy. Therefore, identification of people at earlier time points in the trajectory of CKD, with appropriate management and earlier referral of those who would benefit from specialist kidney services, should lead to both economic and clinical benefits.

Early detection of CKD also allows for more optimal dialysis starts, which are defined as initial therapy with a permanent vascular or peritoneal access or a preemptive transplant. Optimal starts are associated with a 56% reduction in mortality and a 65% reduction in sepsis. Optimal starts were also associated with lower utilization including a 55% reduction in inpatient days. Thus, earlier detection of CKD and referral to nephrology resulted in lower morbidity and mortality associated with dialysis initiation as well as lower utilization of healthcare resources.

The most widely used CKD risk staging system is the “heat map” from KDIGO (Kidney Disease: Improving Global Outcomes) based on estimated glomerular filtration rate (eGFR) and level of albuminuria. This KDIGO guidance is also supported by the National Kidney Foundation-Kidney Disease Outcomes Quality Initiative (NKF-KDOQI). The NKF/American Society of Nephrology (ASN) Task Force has provided updated guidance on estimating GFR, recommending updated equations that remove race from the calculation and indicating that combining filtration markers creatinine and cystatin C is more accurate and supports better clinical decisions. Albuminuria is typically quantified by a urine albumin/creatinine ratio (UACR). Importantly, high levels of proteinuria are associated with an increased risk of disease progression, even if the eGFR is normal. Thus, NKF and ASN recommend measuring both eGFR and UACR for evaluating CKD risk in patients with risk factors, including DM, HTN, and cardiovascular disease. The American Diabetes Association also recommends regular CKD screening and monitoring in patients with DM. The US Preventive Services Task Force is currently reviewing its guidance, with strong advocacy by the NKF and ASN in a joint statement highlighting the urgent need among adults with DM, as only 40% of this important group receive albuminuria screening each year.

However, the KDIGO classification system requires multiple measurements, including both a blood draw and a urine sample. Patients may not have a healthcare provider (HCP), or this may require multiple visits to different settings to obtain each measure. This process can take up to weeks to complete and for the patient to receive the final results. Further, obtaining a KDIGO-based CKD assessment can be costly due to the cost of the HCP visit, laboratory testing, the number of trips required to complete the visit and testing, and time off work, which are clear barriers to many people who need and can benefit from CKD assessment.

It is an object of the present disclosure to address at least one of the foregoing problems or at least to provide the public with a useful choice.

Further aspects and advantages of the present disclosure will become apparent from the ensuing description which is given by way of example only.

### SUMMARY

The present technology provides systems and methods for retinal image analysis using artificial intelligence (AI). Because retinal images, also referred to as fundus images, are routinely taken as part of medical screening procedures (for example, retinal screening for diabetic retinopathy), these images have the potential to be rapidly analysed at low cost for improving chronic kidney disease (CKD) risk prediction, and made available immediately to the patient and their health care provider with no additional burden to the patient.

According to one aspect of the present technology there is provided a method of determining an indication of risk of chronic kidney disease (CKD) of an individual, comprising: determining an indication of risk of chronic kidney disease (CKD) by a deep learning model based on one or more fundus images.

According to one aspect of the present technology there is provided a method, comprising: determining an indication of risk of chronic kidney disease (CKD) by a deep learning model based on one or more fundus images; determining the relative contribution of one or more risk contributing factors to the indication of risk of chronic kidney disease.

In examples, the deep learning model may comprise a plurality of retinal predictor models, each configured to process at least one fundus image of an eye and output at least one feature.

In examples, the deep learning model may comprise at least one CKD risk prediction model configured to receive the features output by the plurality of retinal predictor models.

In examples, the indication of risk may be a two dimensional matrix comprising a plurality of categories of glomerular filtration rate (eGFR), a plurality of categories of albuminuria (ACR).

In examples the risk contributing factors may include two or more of: blood pressure (e.g., systolic blood pressure), glycated haemoglobin A1c (HbA1c), glomerular filtration rate (eGFR), albuminuria (ACR).

In examples, the relative contribution of the one of more of the risk contributing factors may be used to determine one or recommendations for management of the individual's wellbeing. For example, the one or more recommendations may include initiating testing and/or investigation for conditions associated with the one or more risk contributing factors.

In examples the method comprises processing one or more fundus images associated with an individual using a Quality Assurance (QA) set of one or more convolutional neural networks (CNNs) to determine whether the one or more fundus images are of sufficient quality for further processing. In examples the method further comprises processing the one or more fundus images determined to be of sufficient quality for further processing using an eye-identification set of one or more CNNs (eye-ID CNN), to identify the one or more fundus images belonging to a single eye.

In examples, one or more fundus images may be processed in order to predict a risk of chronic kidney disease (CKD) from the one or more fundus images. In examples the method may comprise processing one or more fundus images associated with an individual using a Quality Assurance (QA) set of one or more CNNs to determine whether the one or more fundus images are of sufficient quality for further processing. In examples the method may comprise processing the one or more fundus images determined to be of sufficient quality for further processing using an eye-identification (eye-ID) set of one or more CNNs, to identify the one or more fundus images belonging to a single eye. In examples the method may comprise processing the one or more fundus images using a plurality of risk contributing factor (RCF) sets of one or more CNNs, wherein each RCF set of one or more CNNs is configured to output an indicator of probability of the presence of a different risk contributing factor in each of the one or more fundus images. In examples the method may comprise producing an individual feature vector based on meta-information for the individual, and the outputs of the plurality of RCF sets of one or more CNNs. In examples the method may comprise processing the individual feature vector using a CKD risk prediction neural network model to output a prediction of CKD risk for the individual.

In examples, the one or more fundus images may be processed using a Quality Assurance (QA) set of one or more convolutional neural networks to determine whether the one or more fundus images are of sufficient quality for further processing.

In examples, classifying an image as unsuitable may comprise determining that the image is not directed to a relevant region of an eye of the individual. In examples, determining the image is unsuitable may comprise determining that at least one property of the image is unsuitable. For example, the image may be determined as being over-saturated, underexposed, out of focus, or blurred.

In examples, a notification may be issued warning a user that the one or more fundus images supplied are unsuitable. This enables one or more replacement images to be supplied.

In examples the one or more fundus images may be adjusted prior to processing. In examples, the image adjustment may be normalisation of the images, for example spatial or intensity normalisation. In examples, spatial normalisation may include one or more of: cropping, scaling, and rotation of the one or more fundus images.

In examples, a color balancing process may be performed on the one or more fundus images. In an example, a Gaussian filter may be applied to the one or more fundus images in order to perform color balancing. Image quality, as it pertains to color, can vary significantly between different fundus camera technologies and/or models. Colour balancing reduces the mismatch in images resulting from this, to assist with further processing. In examples, the one or more fundus images may be converted from a colour image into a greyscale or monochrome image.

In examples, a brightness adjustment process may be performed on the one or more fundus images. Image brightness can greatly vary due to environmental conditions (for example, lighting within a clinic) and patient pupil size. Brightness adjustment normalizes these variations to assist with further processing.

In examples in which the one or more fundus images comprises a plurality of fundus images, the plurality of fundus images may be processed using an eye-identification (eye-ID) set of one or more convolutional neural networks configured to group the fundus images as belonging to a single eye—for example, for future clinical results aggregation. In examples the eye-ID CNN operates by identifying an eye as left-eye or right-eye, understanding the “likeness” of several images, and one or more parameters including, but not limited to, image time stamp and patient unique ID. A grouping of images may be referred to as an image set.

In examples, one or more CNNs may be configured to identify a relative location of the one or more fundus images on the retina. For example, the one or more CNNs may be configured to determine whether the one or more fundus images are macula-centred or disk-centred. Two main landmarks of the retina are the macula, which has the densest photoreceptor concentration and is responsible for central vision, and the disk, where the optic nerve enters the eye. In examples, the eye-ID CNNs may be configured to determine if the one or more fundus images are foveal centred. In examples, the eye-ID CNNs may be configured to identify a relative location of the one or more fundus images on the retina.

In examples, one or more CNNs may be configured to determine a device, or characteristic of the device, used to capture the fundus image. In examples the one or more CNNs may be configured to determine whether the device utilises flash photography or white LED confocal photography. In examples, processing of the fundus image may be based at least in part on determination of the device, or the characteristic of the device. In examples, adjustment of the one or more fundus images prior to processing may be based at least in part on the determination of the device, or the characteristic of the device.

In examples, the one or more fundus images are processed by a plurality of risk contributing factor (RCF) sets of one or more CNNs, each RCF set of one or more CNNs configured to output an indication of the probability of the presence of a different risk contributing factor. In examples, the risk contributing factors may include two or more of: blood pressure (e.g., systolic blood pressure), glycated haemoglobin A1c (HbA1c), glomerular filtration rate (eGFR), albuminuria (ACR). In examples, each of the CNNs may produce a probability of an indicator of this risk contributing factor. For example, the CNNs may look for “localized” signs of biological changes and physiological changes (e.g. microaneurysms, oedema, etc.) changes, and or “global” changes in an image that could indicate presence of glycemic control, blood pressure, cholesterol, and exposure to smoking (e.g. pigmentary changes in the peripapillary region, arterial/venous crossing deformations, vascular tortuosity changes, vascular calibre changes, etc.). In examples the signs may include, but not be limited to: drusen appearance, clustering, and/or location; pigmentation change in density and/or location; arteriovenous crossing; change in arteriovenous crossing calibre and/or thickness change; arteriovenous tortuosity; retinal oedema size and/or pattern; and/or microaneurysms concentration.

In examples the plurality of risk contributing factor (RCF) sets of one or more CNNs may be configured to respectively target a plurality of labels selected from the group of: pigmentation abnormalities, drusens, microaneurysms, haemorrhages, eGFR, ACR, HbA1c, systolic blood pressure.

In examples, at least one of the RCF CNNs may be configured in a jury system model comprising a plurality of jury member CNNs, wherein each jury member CNN is configured to output a probability of a different feature in the one or more fundus images, and the outputs of the plurality of jury member CNNs are processed to determine the indicator of probability of the presence of the risk contributing factor output by the RCF CNN.

For example, investigation of each risk contributing factor (e.g. glycaemic control, blood pressure, cholesterol, and exposure to smoking) may include a plurality (for example, at least five) of jury members. Each jury member may be configured to output a probability. The jury system model may produce a final probability based on the outcomes from each jury member. In examples the outputs of the plurality of jury member CNNs may be processed to determine the indicator of probability of the presence of the risk contributing factor output by the RCF CNN based on an expected population baseline for a population to which the individual belongs.

In examples, the outputs from the risk contributing factor (RCF) sets of one or more CNNs are aggregated using minimum, maximum, mean, and median in both model-level and image-level to generate an individual-level fundus image feature vector. In examples, the raw output of each model may be several floating values, where the length of output is model-dependent. The output aggregation firstly happens on a model-level. For example, for an input fundus image, five juror models give probabilities from 0 to 1, i.e. a minimum of 0 and a maximum of 1 (e.g. a decimal value such as 0.01454), and the probabilities for each grade level across five models are also aggregated. In examples, the output of the models are floating-point numbers and after the aggregation using a mathematical operation (including, but not limited to, weighted mean, min, max, etc.), the final output is still in the form of floating numbers. In examples, these floating-point numbers, are concatenated to form a one-dimensional array (i.e. the individual-level fundus image feature vector). In examples, meta-information of an individual associated with the one or more fundus images is combined with the individual-level fundus image feature vector to produce an individual feature vector. In examples a meta-information vector is produced from the meta-information. In examples, the meta-information is pre-processed using one or more of standardisation and one-shot encoding. For example, numerical feature such as age may be standardised to have a mean 0 and standard variance 1. For example, categorical features (e.g. gender and ethnicity) may be converted from string data to numerical vectors using one-shot encoding. In examples, the individual-level fundus image feature vector and the meta-information vector may be concatenated to produce the individual feature vector. This provides a metarepresentation understandable by neural networks.

In examples the CKD risk prediction neural network model utilises a fully connected neural network (FCNN). In examples the FCNN may have at least 5 layers. In examples, the relative contribution of each modifiable factor (e.g. glycaemic control, blood pressure, cholesterol, and exposure to smoking) to the overall CKD risk score is determined. This combination is not an equation, but rather an algorithmic approach, where the patient biometrics are combined and weighted appropriately with their retinal images, within the deeper layers of the overall FCNN design.

In examples, the functionality of two or more of the respective sets of one or more convolutional neural networks disclosed herein may be provided by a single set of one or more convolutional neural networks.

In examples, the system may be configured to report CKD risk on one or more of: an individual level, and a population level. At an individual level, an individual overall CKD risk may be reported—i.e. the overall risk of CKD to an individual associated with processed fundus images. In examples, the system may be configured to report on the contributing factors to the individual overall CKD risk, including non-modifiable contributing factors (e.g. based on patient meta-information such as age, gender, and/or ethnicity) and modifiable contributing factors (e.g. based on blood pressure, HbA1c, eGFR, and albuminuria). In examples the system may be configured to identify the relative contribution of the respective modifiable contributing factors. In examples the system may be configured to rank the modifiable contributing factors according to their relative contribution to the individual overall CKD risk.

At a population level, the system may be configured to report analysis is presented where the overall cohort CKD risk profile and its contributing factors are generated. By way of example, the cohort may be that a population at local, regional, or national levels, the population of a healthcare provider, that of an organisation, or subsets thereof (for example, risk levels within the overall population). Similarly to the individual overall CKD risk, the system may be configured to report on the respective relative contributions of modifiable contributing factors at a population level.

In examples, the system may be configured to provide a recommendation for management of an individual's condition based on the determined risk. For example, a scale of risk levels may be provided, each risk level having an associated recommendation. In examples, at least one recommendation may be provided based on the relative contribution of each modifiable contributing factor. Such recommendations may relate to one or more of: lifestyle (e.g. diet and exercise), further clinical assessments (e.g. cardiologist consultation), or medication (e.g. adherence) decisions.

In examples, the results could be sent to an agency for further analysis, e.g. a healthcare payer for population health analysis.

In examples, the system may be configured to compare at least one of the overall CKD risk, and the relative contribution of each of the risk contributing factors to the overall CKD risk, of the individual to at least a portion of a population of individuals for whom the overall CKD risk is predicted by the CKD risk prediction neural network model, and report an indication of the comparison.

In examples, the system may be configured to predict a change to the overall CKD risk based on a change to one or more of the risk contributing factors. In examples the system may be configured to predict a group overall CKD risk for at least a portion of a population of individuals for whom the overall CKD risk is predicted by the CKD risk prediction neural network model. In examples, the system may be configured to predict a change to the group overall CKD risk based on a change to one or more of the risk contributing factors for at least a portion of the population of individuals.

According to one aspect of the present technology there is provided a computer program product, the computer program product comprising: a non-transitory computer-readable medium having computer-readable program code stored thereon, the computer-readable program code comprising instructions that when executed by a processor, cause the processor to perform a method of determining an indication of risk of chronic kidney disease (CKD) of an individual described herein.

According to one aspect of the present technology there is provided a system comprising a memory storing program instructions; and at least one processor configured to execute program instructions stored in the memory, wherein the program instructions cause the processor to perform a method of determining an indication of risk of chronic kidney disease (CKD) of an individual described herein.

The above and other features will become apparent from the following description and the attached drawings.

### BRIEF DESCRIPTION OF THE DRAWINGS

Further aspects of the present disclosure will become apparent from the following description which is given by way of example only and with reference to the accompanying drawings in which:

FIG. 1 is a schematic diagram of a system depicting various computing components that can be used alone or together in accordance with aspects of the present technology; and

FIG. 2A shows a diagram of an exemplary architecture for an ensemble of neural networks utilised in accordance with aspects of the present technology.

FIG. 2B is a diagram showing a jury model structure of a set of CNNs used in the system in accordance with aspects of the present technology.

FIG. 3 is a map of CKD risk by eGFR and albuminuria categories from KDIGO (Kidney Disease: Improving Global Outcomes).

FIG. 4A shows a diagram of another exemplary architecture for an ensemble of neural networks utilised in accordance with aspects of the present technology.

FIG. 4B is a block diagram illustrating the architecture of a maculopathy predictor model.

FIG. 4C is a block diagram illustrating the architecture of a retinopathy predictor model.

FIG. 4D is a block diagram illustrating the architecture of a retinal age predictor model.

FIG. 4E is a block diagram illustrating the architecture of an Albumin-to-Creatinine Ratio predictor model.

FIG. 4F is a block diagram illustrating the architecture of an Estimated Glomerular Filtration Rate predictor model.

FIG. 4G is a block diagram illustrating the architecture of a Systolic Blood Pressure predictor model.

FIG. 4H is a block diagram illustrating the architecture of a Total Cholesterol predictor model.

FIG. 4I is a block diagram illustrating the architecture of a Hemoglobin A1C predictor model.

FIG. 4J is a block diagram illustrating the architecture of a race predictor model.

FIG. 4K is a block diagram illustrating the architecture of a smoking status predictor model.

FIG. 4L is a block diagram illustrating the architecture of a cardiovascular risk biomarker velocity predictor model.

FIG. 4M is a block diagram illustrating the architecture of a KDIGO based CKD risk prediction model.

FIG. 4N is a block diagram illustrating the architecture of a Chronic Kidney Disease Prognosis Consortium based CKD risk prediction model.

FIG. 4O is a block diagram illustrating the architecture of a Kidney Failure Risk Equation based CKD risk prediction model.

FIG. 4P is a block diagram illustrating the architecture of a Framingham Risk Score based CKD risk prediction model.

FIG. 4Q is a block diagram illustrating the architecture of an Extended Framingham Risk Score based CKD risk prediction model.

FIG. 4R is a block diagram illustrating the architecture of a Framingham CKD Risk based CKD risk prediction model.

FIG. 5 shows a diagram of an exemplary architecture for a system implementing aspects of the present technology.

### ACRONYMS

Reference may be made herein to terms using acronyms as set out in the table below:

| | |
|---|---|
| ACR | Albumin-to-Creatinine Ratio |
| ASCVD | Atherosclerotic cardiovascular disease |
| CKD | Chronic Kidney Disease |
| CKD-F | Framingham CKD risk |
| CKD PC | Chronic Kidney Disease Prognosis Consortium |
| DM | Diabetes (type 2) |
| eGFR | Estimated Glomerular Filtration Rate |
| EX-FRS | Extended Framingham Risk Score |
| FRS | Framingham Risk Score |
| HbA1C | Hemoglobin A1C |
| HF | Heart failure |
| HTN | Hypertension |
| KDIGO | Kidney Disease: Improving Global Outcomes |
| KFRE | Kidney Failure Risk Equation |
| M | Maculopathy |
| R | Retinopathy |
| RFI | Retinal Fundus Image |
| SBP | Systolic Blood Pressure |
| T Chol | Total Cholesterol |

### DETAILED DESCRIPTION

The present technology is generally directed to determining a person's risk of chronic kidney disease (CKD) using their retinal photographs (also known as fundus images) and demographic information (e.g., details such as age, gender and ethnicity) with artificial intelligence technology. These images and information may be obtained by optometrists, for example, as part of a routine eye examination.

The human eye offers a unique window into the body's overall health, as it reflects the condition of various internal organs, including the vascular system. The fact that the retina can reflect kidney, metabolic, and cardiovascular health and disease has been known for more than 6 decades. However, only the new technological advances of AI and machine learning for retinal fundus image (RFI) analysis, plus broad availability of non-mydriatic retinal cameras, have made it possible for automated RFI-based risk and disease detection at scale. More than 50,000 eye clinics in the United States, distributed throughout both urban and rural communities, are currently equipped with non-mydriatic retinal cameras.

RFI involves capturing high-resolution images of the retina, which is the layer of tissue at the back of the eye responsible for vision. These images provide valuable information about the health of the eye, including the presence of abnormalities such as retinal detachment, diabetic retinopathy, hypertensive retinopathy, macular degeneration, and glaucoma. Traditionally, ophthalmologists and optometrists have relied on their clinical expertise to interpret these images manually. However, this limits availability and the process can be time-consuming, subjective, and prone to human error.

RFI may provide one or more advantages. Non-invasiveness: One of the significant advantages of using RFI is the non-invasive nature of the procedure. Unlike traditional diagnostic methods that may require invasive procedures or exposure to ionizing radiation, RFI allows for a painless and risk-free examination. This non-invasiveness reduces patient discomfort and helps promote use.

Early disease detection: By examining the retinal images, clinicians can detect early signs of various systemic diseases, such as DM, HTN, kidney disease, cardiovascular diseases, and even some forms of cancer. Early detection enables timely intervention and treatment, improving patient outcomes and reducing healthcare costs.

Accuracy: RFI provides highly detailed and precise information about the structure and condition of the retinal blood vessels, optic nerve, and other retinal components. These images offer valuable insights into the microvascular changes and abnormalities associated with multiple diseases. By analyzing these images, AI algorithms can identify subtle patterns, biomarkers, and abnormalities that may not be visible to the eye on clinician examination, promising to enhance diagnostic accuracy.

Wide accessibility: RFI can be performed using non-mydriatic cameras, meaning that they do not require the pupils to be dilated. These cameras are compact, portable, and cost-effective. This accessibility allows for broader risk assessment programs in primary care settings, community health centers, and even remote areas with limited resources. This ease of use makes RFI a valuable tool for risk and disease detection in underserved populations.

Affordability: Retinal imaging is inexpensive compared to traditional CKD assessment, which typically requires multiple laboratory tests and in-person visits to a healthcare professional. People from lower socio-economic backgrounds frequently receive an eye exam due to need for prescription glasses, where retinal imaging is, or can be, performed routinely. This provides a unique opportunity for an affordable and point-of-care CKD assessment.

AI can analyze retinal fundus images and provide valuable insights to aid in the diagnosis and monitoring of eye conditions. AI algorithms have been developed to detect and classify various abnormalities, such as the presence of microaneurysms and hemorrhages in diabetic or hypertensive retinopathy, drusen deposits in macular degeneration, and optic nerve damage in glaucoma.

One of the significant advantages of AI is its ability to process images rapidly. AI algorithms can analyze a large number of retinal images in a short amount of time, allowing for high-throughput screening and population-level analysis. This scalability is particularly beneficial in areas with limited access to eye care services, where AI can assist in triaging patients and identifying those in need of attention. By automating the initial risk assessment process, AI can help alleviate the burden on healthcare systems, broaden access, and ensure that resources are allocated effectively.

Moreover, AI algorithms applied to RFI can also predict the progression and prognosis of diseases. By analyzing large datasets of retinal images and patient records, AI can identify patterns and risk factors that may contribute to disease progression. This predictive capability can help clinicians intervene at an early stage and provide personalized treatment plans to patients. For instance, AI algorithms can identify individuals at high risk of developing glaucoma based on specific features in their retinal images, allowing for timely interventions and preventive measures. The integration of AI with RFI has led to the development of computer-aided diagnosis (CAD) systems. These systems can assist healthcare providers in making more informed decisions.

Risk factors for CKD, such as DM, HTN, and cholesterol emboli, can often manifest in the eye. As the blood vessels can be non-invasively visualized from RFI, various features in the retina may reflect systemic health as well as future risk. The retina is unique being the only part of the human vasculature that is visible by non-invasive means. In recent years there has been an exponential increase in the number of studies that have used AI, and deep learning (DL) in particular, to extract data from retinal images. Having recognized the power of DL to extract data from retinal images there is now intense interest in using the retinal image data generated by DL algorithms to augment the traditional means of estimating CKD risk.

A significant limitation to prior AI models for CKD has been their focus on eGFR rather than the guideline standard of KDIGO, which is based on both eGFR and UACR. Thus, in developing the present technology, UACR data has been incorporated into the training of the models, and then tested against the KDIGO standard.

Aspects of the present technology provide a deep learning (DL) model that uses retinal photographs and limited demographic data to classify an individual's CKD risk.

In summary, the present technology offers the potential to significantly improve access to individualised recommendations for wellbeing—and in particular CKD risk prevention strategies—as the risk predictions they produce do not require either clinical or laboratory assessments to generate an individual's risk. As retinal photographs are routinely captured in optometric practices it means the technology can be deployed without significant additional investment in primary care, a feature which makes these technologies particularly relevant to low-resource settings. Finally, AI-based prediction tools that assess risk at the individual level would inform treatment decisions based on the specific needs of an individual, thereby increasing the likelihood of positive health outcomes.

### 1. System

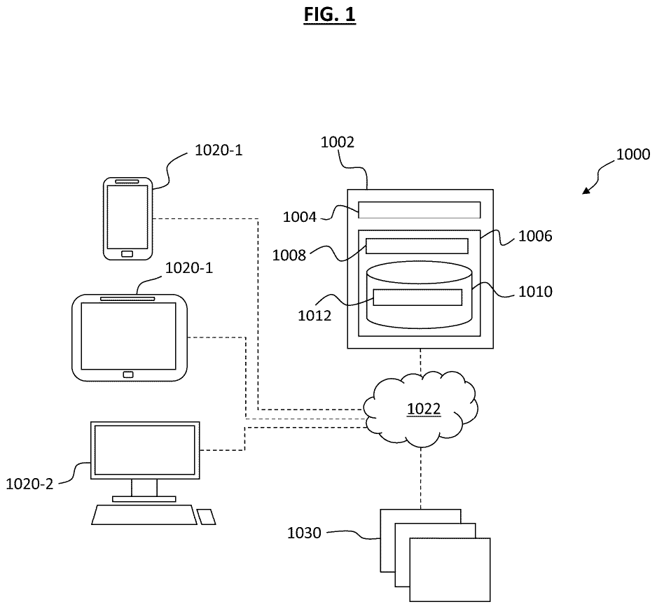

FIG. 1 presents a schematic diagram of a system 1000 depicting various computing components that can be used alone or together in accordance with aspects of the present technology. The system 1000 comprises a processing system 1002. By way of example, the processing system 1002 may have processing facilities represented by one or more processors 1004, memory 1006, and other components typically present in such computing environments. In the exemplary embodiment illustrated the memory 1006 stores information accessible by processor 1004, the information comprising instructions 1008 that may be executed by the processor 1004 and data 1010 that may be retrieved, manipulated or stored by the processor 1004. The memory 1006 may be of any suitable means known in the art, capable of storing information in a manner accessible by the processor, comprising a computer-readable medium, or other medium that stores data that may be read with the aid of an electronic device. The processor 1004 may be any suitable device known to a person skilled in the art. Although the processor 1004 and memory 1006 are illustrated as being within a single unit, it should be appreciated that this is not intended to be limiting, and that the functionality of each as herein described may be performed by multiple processors and memories, that may or may not be remote from each other.

The instructions 1008 may comprise any set of instructions suitable for execution by the processor 1004. For example, the instructions 1008 may be stored as computer code on the computer-readable medium. The instructions may be stored in any suitable computer language or format. Data 1010 may be retrieved, stored or modified by processor 1004 in accordance with the instructions 1008. The data 1010 may also be formatted in any suitable computer readable format. Again, while the data is illustrated as being contained at a single location, it should be appreciated that this is not intended to be limiting—the data may be stored in multiple memories or locations. The data 1010 may comprise databases 1012.

In some embodiments, one or more user devices 1020 (for example, a mobile communications capable device such as a smartphone 1020-1, tablet computer 1020-2, or personal computer 1020-3) may communicate with the processing system 1000 via a network 1022 to gain access to functionality and data of the processing system 1002. The network 1022 potentially comprises various configurations and protocols comprising the Internet, intranets, virtual private networks, wide area networks, local networks, private networks using communication protocols proprietary to one or more companies—whether wired or wireless, or a combination thereof. For example, fundus images obtained from one or more fundus imaging devices (herein referred to as a “fundus camera” 1030) may be input to the processing system 1002 via the user devices 1020.

A fundus camera typically comprises an image capturing device, which in use is held close to the exterior of the eye and which illuminates and photographs the retina to provide a 2D image of part of the interior of the eye. Many clinically important regions of the eye may be imaged, comprising the retina, macula, fovea, and optic disc. A single fundus image of a non-dilated eye captures less than 45° of the back of the eye. In practice, a clinician will often choose to capture several photographs while guiding the patients to look up, down, left and right, to create a larger field of view of the retina.

### 2. First Exemplary Model

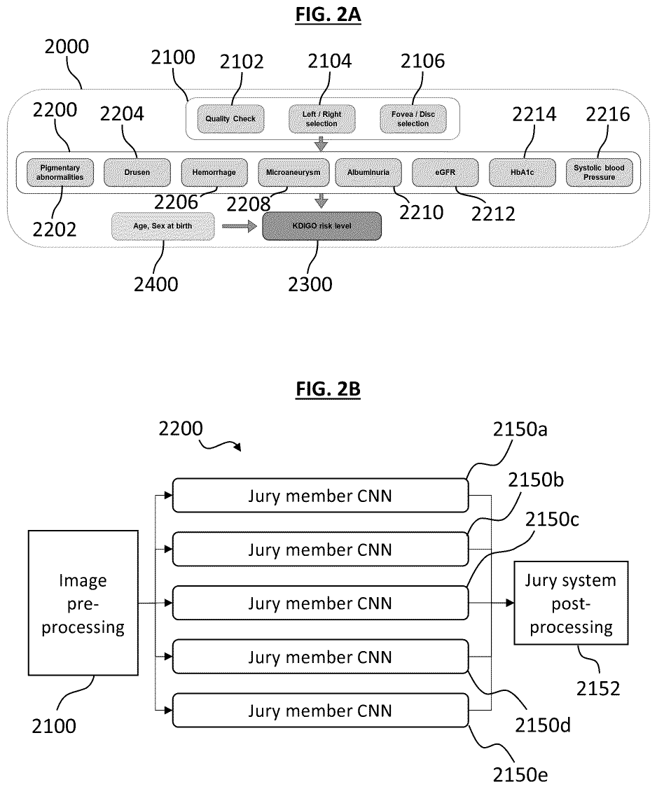

FIG. 2A shows an architecture for an ensemble of DL models 2000, classified into two primary categories: (1) image-based models, including first image-based models 2100 and second image-based models 2200, and (2) non-image-based model 2300. The imaged based models 2100 and 2200 use image data as input and includes models for detection of microaneurysms, drusens, changes in vascular tortuosity, etc. (as described further below). The non-image-based model 2300 relies on vectorized interpretations of participant information including their biometrics, which combines demographic data 2400 (e.g., patients age and sex at birth) with the vector output of second image-based models 2200, to create a single vector representing the totality of learnings from the image, age, and sex of the patient.

Together, these models are focused on different aspects of CKD risk (including but not limited to abnormal eGFR, albuminuria, high blood pressure, elevated blood sugar level) and their unique representation in the retina. The outputs of these models are then combined, while considering age and sex at birth, to create the KDIGO classification results, serving as the target binary output of elevated CKD risk or not.

The full model then takes images and non-image data (age and sex at birth) to produce the final binary outcome. The present technology relies solely on retinal images and patient demographics (e.g., age and sex at birth), obtained by a healthcare provider (HCP), which is easily accessible, non-invasive, and can be captured in a matter of seconds.

For completeness, it will be appreciated that the deep learning models and frameworks disclosed herein are provided by way of example, and that viable alternatives will be apparent to the skilled addressee. The model 2000 utilises various convolutional neural networks (“CNN”). CNNs are deep learning architectures particularly suited to analysing visual imagery. A typical CNN architecture for image processing consists of a series of convolution layers, interspersed with pooling layers. The convolution layers apply filters, learned from training data, to small areas of the input image in order to detect increasingly more relevant image features. A pooling layer down-samples the output of a convolutional layer to reduce its dimensions. The output of a CNN may take different forms depending on the application, for example one or more probabilities or class labels.

The various levels of the model 2000 of FIG. 2A are described below.

### 2.1 First Level

The first level 2100 includes an image quality control CNN (QC) 2102, a laterality (Left eye/Right eye) detector CNN 2104, and an image location (fovea/non-fovea) detector CNN 2106. The input of this layer is fundus images only. This process ensures that only foveal centered images that are of sufficiently high quality are accepted into the model. Identifying the laterality of the image assists with aggregation of all images of each eye for each individual during the analysis.

### 2.1.1 QC CNNs

The QC CNNs 2102 are trained by inputting sample images previously labelled by an expert clinician, and training them for sufficient iterations. In an example, a QC CNN was based on a modified XCEPTION design (although it is noted that a modified Inception-ResNet-v2 design may be utilised), and trained using a dataset of 20,000 images, wherein the dataset comprised similar proportions of four types of images: Type 1: Eyeballs, rooms or other irrelevant images; Type 2: Severely over-saturated or underexposed images; Type 3: Less than perfect images that could still be useful to a clinician in conducting a manual analysis; and Type 4: High quality images.

Experiments were run in an Intel Xeon Gold 6128 CPU @ 3.40 GHz with 16 GB of RAM memory and a NVIDIA Geforce TiTan V VOLTA 12 GB on Windows 10 Professional. Tensorflow 1.11.0 and Python 3.6.6 were utilised to implement the QA CNN 3004 models.

Hyperparameters comprised: (i) Batch Size: 64. Batch size refers to the number of training samples utilised in one step. The higher batch size, the more memory space need. For an input image size of 320*320, and GPU memory of 12 GB, the batch size was set at 64; (ii) Training\validation\testing split: (70\15\15); (iii) Epoch: 100. One epoch refers to one forward pass and one backward pass of all the training examples; (iv) Learning algorithms: the ADAM optimizer was utilised, being an advanced version of stochastic gradient descent; (v) Initial Learning Rate: 10e-3. Learning rate controls how much model adjusting the weights with respect the loss gradient. Typical learning rates are in the order of [10e-1, 10e-5]. In view of use of the ADAM optimizer and batch normalization, the initial learning rate was initially set at 10e-3; (vi) Loss Function: Softmax Cross Entropy; (vii) Dropout rate: 0.5.

The QA CNN described above achieved 99% accuracy in classifying an input image to the categories. Following training, all of the Type 1 and 2 images were removed. Type 3 images are shown to the clinician, but are not used in further processing. Type 4 images are used as part of further processing.

In examples, one or more lighting type CNNs may be configured to determine a device, or characteristic of the device, used to capture the input fundus image. There are two main photography technologies for fundus imaging: a) flash photography, and b) white LED confocal photography, which produce different looking images. Depending on the camera source (and therefore lighting of the image, the subsequent processing may be adjusted.

### 2.1.2 Eye-ID CNNs

Clinicians often obtain more than one image from a single eye, creating a larger view of the back of the eye. A set of eye-identification (eye-ID) CNNs including laterality detector CNN 2104, and image location detector CNN 2106, are trained to find similarities between several viewpoint images of the same eye, and group them into a single image set. It is important to identify images that belong to the same eye, as a final clinical outcome may be the sum of analysis of each single image in that set.

An exemplary training environment for the eye-ID CNNs is similar to that described above for the QA CNNs. A database of 160,585 images, from 75,469 eyes of 40,160 people was created. Each image was labelled with Left/Right eye, patient ID (when available) and time stamp of image acquisition. The laterality detector CNN 2104 were trained on this data set to identify the orientation (Left/Right) of images, and group them based on ID/acquisition time. The trained laterality detector CNN 2104 achieved more than 99% accuracy. The image location detector CNN 2106 is further trained to identify the location of the images on the retina, including identifying the location as being macula-centered or disk-centered. This enables selection of foveal centered images for further processing.

When implemented, the eye-ID CNNs group multiple images submitted by clinician into eye and patient subgroups.

### 2.1.3 Image Preparation

In examples, the fundus images may also be adjusted before further processing—for example by performing brightness adjustment and colour balancing for normalisation purposes, and cropping and scaling the images for standardisation.

In an example, a Gaussian filter may be applied to the original fundus photo. An example of such a filter may be expressed as:

$${I}_{c} = α I + β G ( ρ ) * I + γ$$

where * denotes the convolution operation, I denotes input image and G(ρ) represents the Gaussian filter with a standard deviation of ρ. While it will be appreciated that parameters may be optimised for each dataset, an exemplary set of parameters may comprise: alpha=4±1, beta=−4±1, gamma=128±50, ratio=10±10.

### 2.2 Second Level

The second level 2200 includes eight ensembles of Als, each consisting of a plurality of CNNs (50 in total) which were trained against various labels in the UK BioBank. Each ensemble targeted a unique label in the fundus images, estimating the following variables: 1. Macular Pigmentation Abnormalities CNNs 2202—the existence and extent of pigmentation abnormalities; 2. Drusen CNNs 2204—the existence and clustering of drusens; 3. Haemorrhage CNNs 2206—the existence and size of haemorrhages; 4. Microaneurysm CNNs 2208—the existence and size of microaneurysms; 5. Albuminuria CNNs 2210—changes related to Albuminuria (ACR); 6. eGFR CNNs 2212—changes related to eGFR; 7. HbA1c CNNs 2214—changes related to HbA1c elevation; and 8. Systolic Blood Pressure CNNs 2216—changes related to systolic blood pressure elevation.

These CNNs follow modified versions of the Inception-Resnet-V2 or ResNet50 structures. Taking the single haemorrhage CNN model as an example, the model has a deep structure, consisting of 164 layers, and uses a combination of inception and residual blocks. The inception blocks use a combination of convolutional layers with different filter sizes, while the residual blocks use skip connections to enable the model to learn from previous layers. Batch normalization and bottleneck layers are employed to improve training efficiency.

### 2.2.1 Jury Model

In examples, a jury system (as described below) may be implemented in the second level 2200 to arrive at a prediction for each biomarker. To elaborate using the retinopathy model as an example, there exist six distinct levels of retinopathy (R0-R5). Five jury models are employed to assess each eye, resulting in 30 probability values per eye. These probabilities are merged and consolidated for both eyes, thereby yielding a final value for each patient.

In examples the second level 2200 CNNs each operate as a “jury” system. Referring to FIG. 2B, each second level 2200 CNN (e.g., Macular Pigmentation Abnormalities CNNs 2202, Drusen CNNs 2204, Haemorrhage CNNs 2206, Microaneurysm CNNs 2208, Albuminuria CNNs 2210, eGFR CNNs 2212, HbA1c CNNs 2214, Systolic Blood Pressure CNNs 2216—referred to generically as a second level 2200 CNN) comprises a plurality of jury member CNNs 2150 (in this example, five jury member CNNs 2050a to 2050e), each jury member CNN 2150 configured to produce a probability of the feature it is trained to look at.

These CNNs follow modified versions of the Inception-Resnet-V2 or ResNet50 structures. Taking the single retinopathy CNN model as an example, the model has a deep structure, consisting of 164 layers, and uses a combination of inception and residual blocks. The inception blocks use a combination of convolutional layers with different filter sizes, while the residual blocks use skip connections to enable the model to learn from previous layers. Batch normalization and bottleneck layers are employed to improve training efficiency. Overall, the model architecture is designed to extract features at multiple scales and capture fine-grained details in images, making it well-suited to detect the level of biomarkers. For each CNN, the dataset was split for training, validation, and testing in 70%, 15% and 15% respectively. The excessive background of the fundus images was cropped, and the resulting image was resized to 800×800 pixels. A batch size of 8 was chosen to optimize GPU memory during training. Adam optimizer was adopted with a learning rate 1*10e-3 to update parameters towards the minimization of the loss. Dropout was enabled with a rate p=0.2, and the model was trained for at least 100 EPOCHs. All codes related to this work were implemented using Python 3.7.

In jury system post-processing 2152, the outcome of each jury member CNN 2150 is then considered, equally or non-equally weighted compared to the rest of the members, to create a statistical representation of the possibility of the changes observed in the particular fundus image.

After receiving the raw outputs from the second level 2200 CNNs, the output results are aggregated. For example, for an input fundus image, five juror models give probabilities from 0 to 1, i.e. a minimum of 0 and a maximum of 1 (e.g. a decimal value such as 0.01454), and the probabilities for each grade level across five models are also aggregated. In examples, the output of the models are floating-point numbers and after the aggregation using a mathematical operation (including, but not limited to, weighted mean, min, max, etc.), the final output is still in the form of floating numbers concatenated to form a one-dimensional array (i.e. the individual-level fundus image feature vector).

### 2.3 Third Level-CKD Risk Prediction

The third level 2300 is a Multi-Layer Perceptron (MLP), which uses the output of the second level 2200 CNNs, plus the patient's demographic data 2400 (e.g., chronological age, and sex at birth), to predict CKD risk.

This CKD risk score is the ground truth label, calculated from nine fields in the UK BioBank dataset for each participant. The architecture of the model comprises an input layer, followed by five dense layers that exhibit a gradual decrease in neuron counts, namely 1024, 512, 256, 128, and 32. These layers are interspersed with batch normalization and LeakyReLU activation functions with a leaky rate of 0.1. To address overfitting concerns, dropout layers with a rate of 0.3 were incorporated after the third, fourth, and fifth dense layers. The ultimate layer, encompassing a single neuron and a linear activation function, predicts the target value. For optimization purposes, an Adam optimizer is utilized with an exponentially decaying learning rate schedule, initialized at 3e-3 and decaying by a factor of 0.95 every 1000 steps. The Huber loss function was employed to guide the model parameters updating. To curb overfitting and ensure efficient training, early stopping was implemented.

In examples, Shapley Additive Explanations (SHAP) algorithm may be used to elucidate the contributions of each variable to the final prediction of CKD risk. This methodology offers a unified approach for the interpretability in machine learning, fostering a comprehensive and integrative understanding of feature importance in predictive models. Inspired by cooperative game theory, the SHAP values elucidate the equitable distribution of contributions across features for each prediction, thereby facilitating the attribution of each feature's influence on the predicted outcome. In contrast to other local interpretable model-agnostic explanations, SHAP values attribute contributions in a consistent manner, adhering to the principles of local accuracy, missingness, and consistency. This ensures that the cumulative attributions align with the total effect.

### 2.4 CKD Risk Results Presentation

In examples, the CKD risk may be presented as a binary outcome—i.e., elevated or non-elevated risk. However, in alternative examples the CKD risk may be determined on a scale, whether a continuous value, or a plurality of grades between low to high risk.

In examples, the CKD risk may be capable of being broken down into the contributing factors, including non-modifiable contributing factors (e.g. based on patient meta-information such as age, gender, and/or ethnicity) and modifiable contributing factors (e.g. based on glycaemic control, or blood pressure). In one example, this is achieved by individual or group analysis of the relative contribution of CNNs that are responsible for the effects of each factor, including an inclusion/exclusion analysis, weight adjustment analysis, and sensitivity analysis.

2.5 Training of Exemplary Model

The model was trained using a dataset of retinal images and health data from >100,000 people combining UK Biobank and the EyePACS 10K datasets. The demographic and risk factor makeup of this training dataset are described in Table 1-1.

TABLE 1-1
The demographic and risk factor makeup of
the training dataset used for the model

| | N = 106,595 people | |
|---|---|---|
| Training Set | Mean | Std |
| Age (years) | 67 | 11 |
| Sex at Birth | 52,673 (49.4%) F | 53,922 (50.6%) M |
| Systolic Blood Pressure (mmHg) | 134 | 17.6 |
| Diastolic Blood Pressure | 80.5 | 11.6 |
| (mmHg) | | |
| HbA1c (%) | 7.1% | 1.8% |
| Total cholesterol(mg/dL) | 209 | 49 |
| HDL cholesterol(mg/dL) | 53.0 | 14.8 |
| eGFR (mL/min) | 91.2 | 26.0 |
| UACR (mg/mmo) | 6.8 | 3 |
| BMI | 29.3 | 7.0 |
| Current Smoker | TRUE | FALSE |
| | 4,106 (9.4%) | 39,392 (90.6%) |

| | Hispanic | Black | Asian | White |
|---|---|---|---|---|
| Race/Ethnicity | 7.3% | 1.9% | 22.4% | 47.9% |

The model was then tested on a separate subset of the US-based EyePACS 10K dataset not previously utilized in the training phase. The EyePACS 10K dataset predominantly comprises individuals (>99%) living with diabetes who are undergoing screening for diabetic retinopathy. Detailed demographic information and risk factor data pertaining to this test subset are presented in Table 1-2.

TABLE 1-2
The demographic and risk factor makeup of the
EyePACS 10K test dataset used for the model

| | N = 4,033 people | |
|---|---|---|
| EyePACS 10K Test Set | Mean | Std |
| Age (years) | 56 | 10 |
| Sex at Birth | 2,239 (55.5%) F | 1,794 (44.5%) M |
| Systolic Blood Pressure (mmHg) | 131 | 12.4 |
| Diastolic Blood Pressure (mmHg) | 71.2 | 8.6 |
| HbA1c (%) | 8.2% | 1.8% |
| Total cholesterol(mg/dl) | 179 | 42.9 |
| HDL cholesterol(mg/dl) | 46.3 | 12.1 |
| eGFR (mL/min) | 97.8 | 23.0 |
| UACR (mg/mmo) | 16.9 | 11.3 |
| BMI | 33 | 11.5 |
| Current Smoker | TRUE | FALSE |
| | 237 (9.9%) | 2,163 (90.1%) |

| | Hispanic | Black | Asian | White |
|---|---|---|---|---|
| Race/Ethnicity | 62.8% | 7.3% | 8.1% | 6.9% |

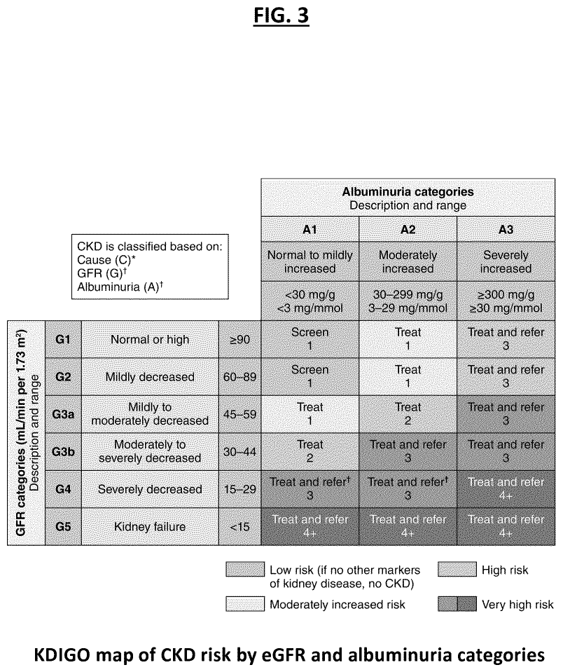

KDIGO classification was determined by eGFR and albuminuria levels (see, FIG. 3). The eGFR was calculated by using the two-factor 2021 CKD-EPI equation (eGFRcr-cys). The serum creatinine and cystatin-C were measured on the same visit as when the retinal images were acquired. UACR was calculated from a spot urine sample by dividing urine albumin by urine creatinine (enzymatic).

In the training set, 74,302 (69.7%) individuals had a low KDIGO risk level and 32,293 (30.3%) individuals had moderate or greater KDIGO risk level. In the EyePACS 10K test set, 2,496 (61.9%) individuals had a low KDIGO risk level and 1,537 (38.1%) individuals had moderate or greater KDIGO risk level. The resulting performance of the exemplary model on the test set for the binary classification of elevated CKD risk was AUC of 83%, sensitivity of 80%, and specificity of 85%.

### 3. Second Exemplary Model

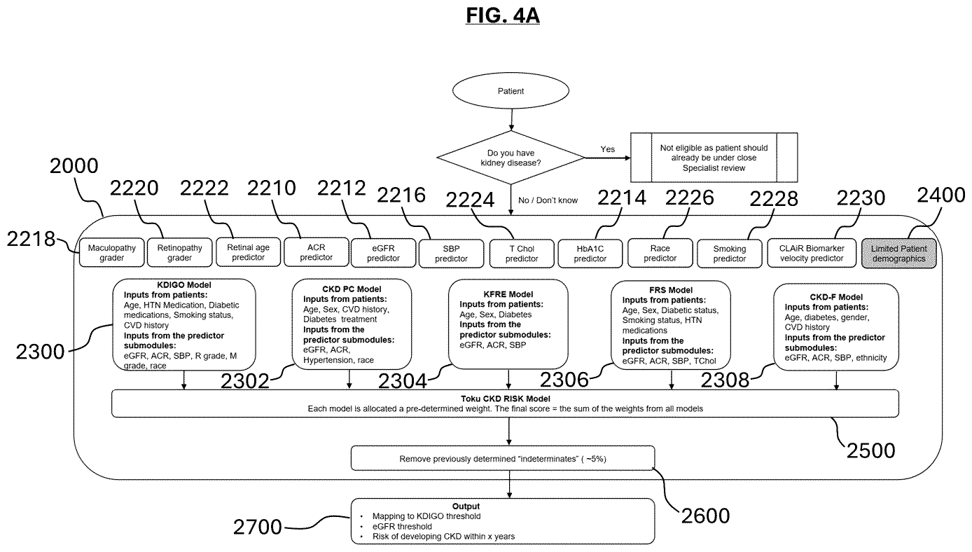

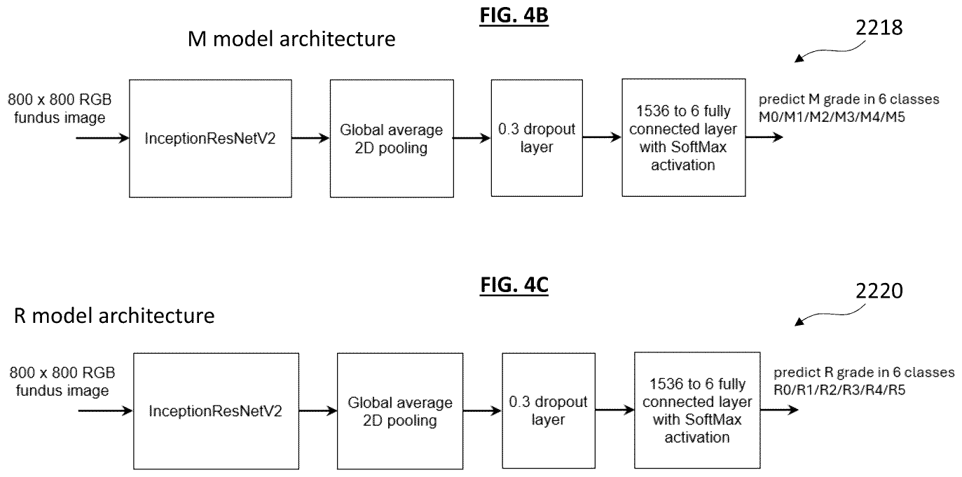

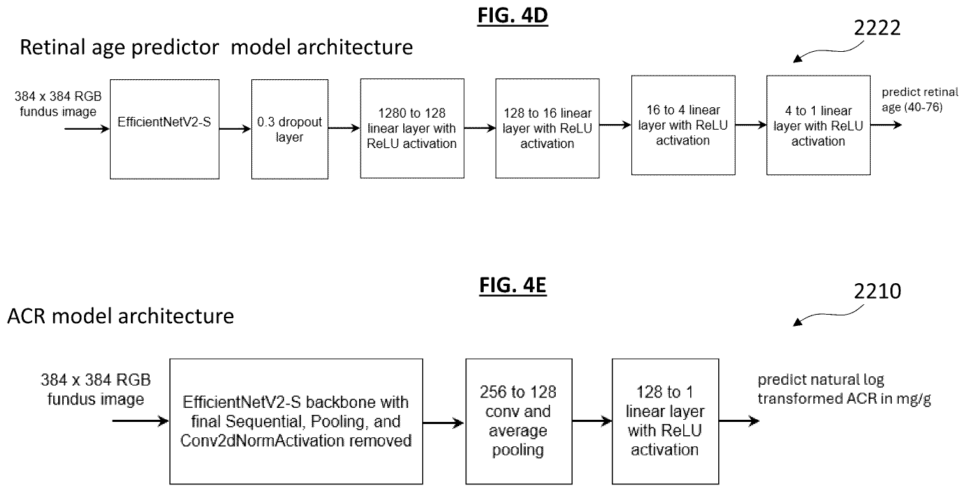

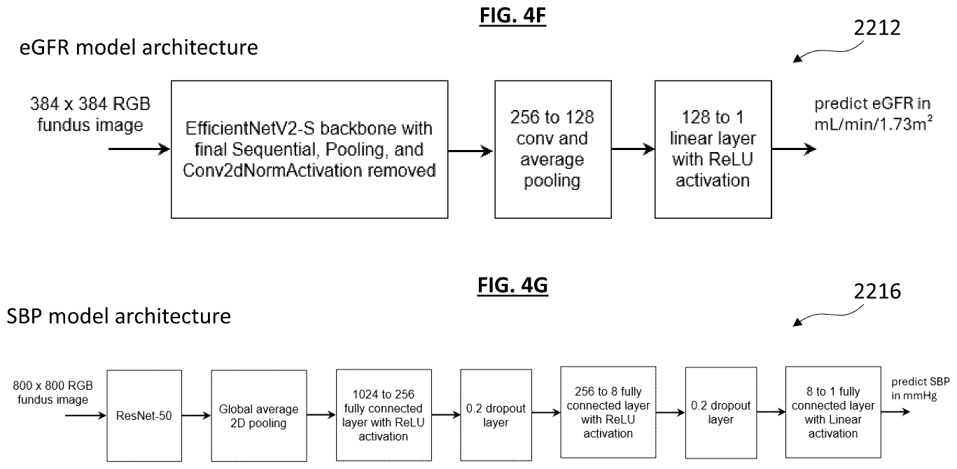

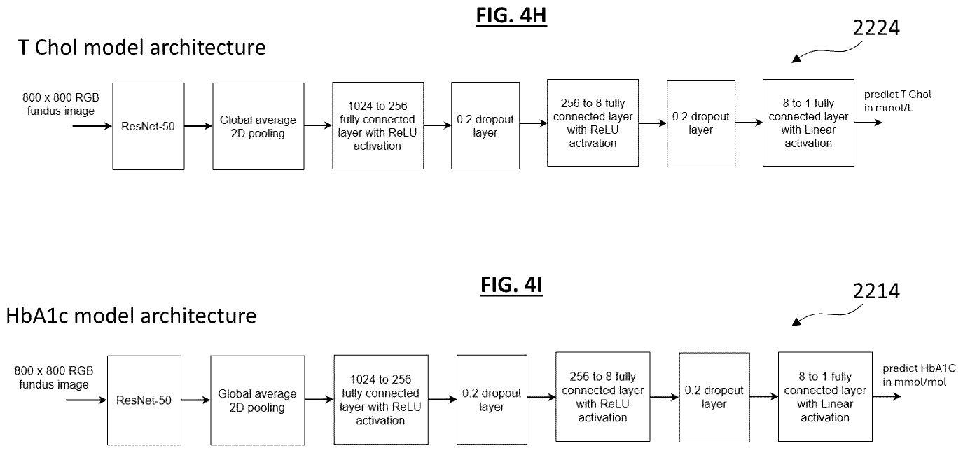

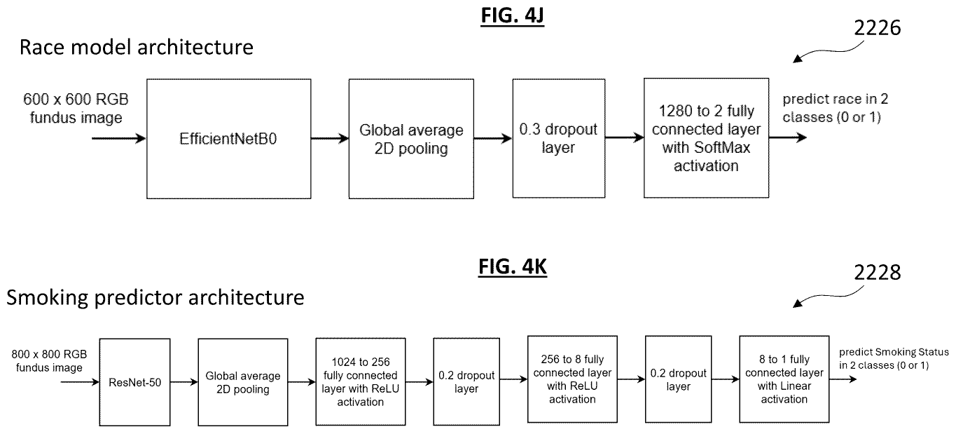

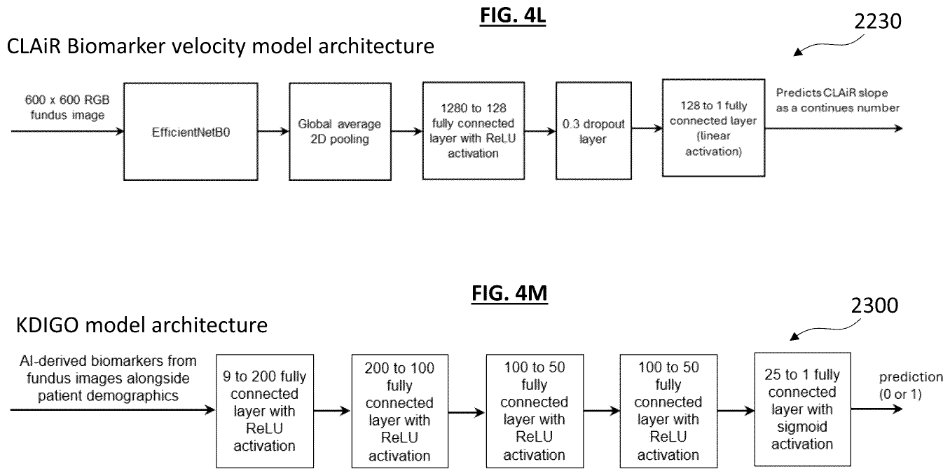

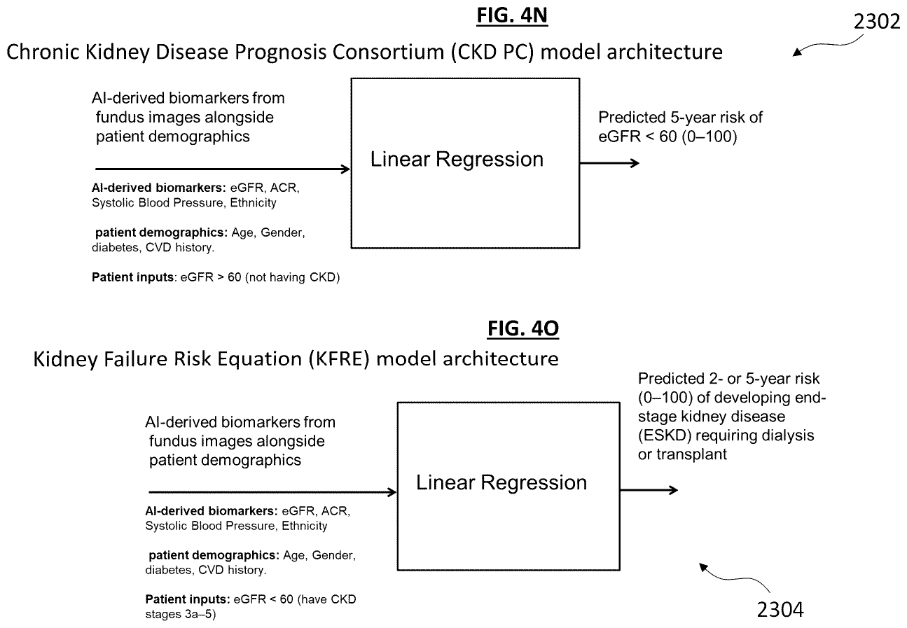

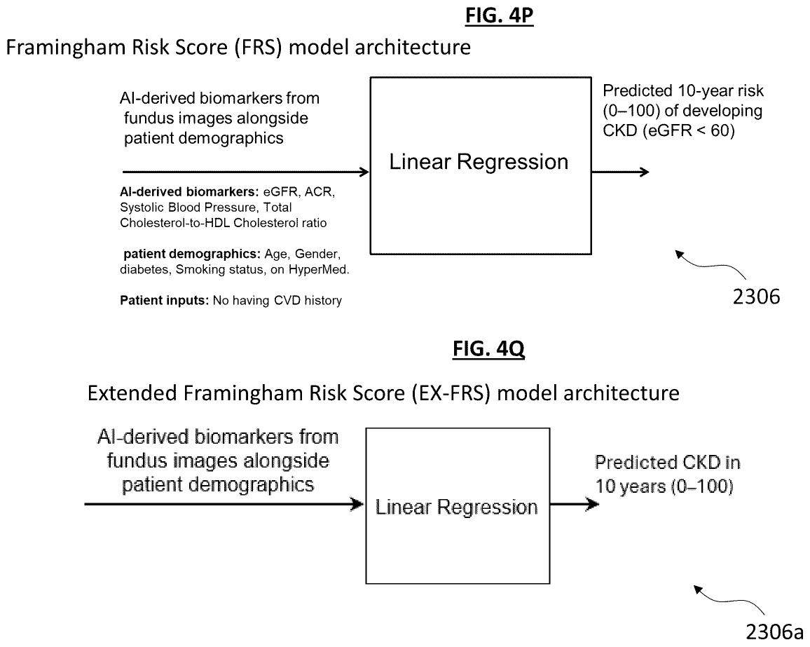

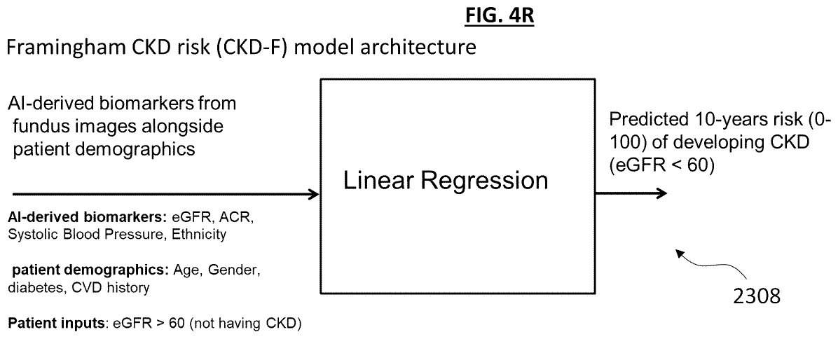

FIG. 4A illustrates another exemplary model 2000 for the prediction of CKD risk. As described above, retinal images may be pre-processed (e.g., using image quality control CNN (QC) 2102, laterality (Left eye/Right eye) detector CNN 2104, and image location (fovea/non-fovea) detector CNN 2106 of FIG. 2A, and/or image preparation as described above).

The exemplary model 2000 uses inputs from eleven retinal predictor submodules (or CNN ensemble)—each of which derive a unique signal from the input retinal image—and limited patient demographics (such as age and sex at birth). The outputs of these twelve sub components serve as the inputs for the CKD risk prediction models (five in the illustrated example, although in alternative examples only one, or subsets of the five, may be implemented). Although the inputs to the five CKD risk prediction models differ slightly (see below for more details), it is envisaged that 80 to 90% of the weighting may be derived from the retinal image based predictor models, and 10 to 20% of the weighting may come from limited patient demographics.

It should be appreciated that all embodiments of the present technology may utilize all eleven AI-derived features. It is envisaged that eGFR, uACR, SBP, R grade and M grade may be considered a first-priority AI-derived feature. In examples, race may be considered a second-priority AI-derived feature. In examples, T Chol, Retinal Age, Smoking status, and CVD risk biomarker velocity may be considered may be considered a third-priority AI-derived feature.

In examples, the patient demographics may include: Age, Gender, diabetic status, Ethnicity, smoking status, CVD history, and hypertension medications (or sub-sets thereof).

In examples, a final prediction of CKD risk 2700 may be generated by a weighted sum 2600 of two or more of the CKD prediction models. In examples, an equal weighting may be applied to each contribution. However, it is envisaged that (in the example of five prediction models being implemented), this weighting may be calibrated in accordance with the population the tool is to be deployed in, and the weighting of each model may be tuned to contribute between 10-30% of the final prediction depending upon the target population being tested.

In examples, it is envisaged that priority weighting may be given as follows: CKD-F model 2308>FRS model 2306>CKD-PC model 2302>KFRE model 2304>KDIGO model 2300. However, it should be appreciated that this is not intended to be limiting to all examples of the present technology. For example, the risk prediction may be determined using a combination of one or more of the models, i.e.: CKD-F; FRS; CKD-PC; KFRE; KDIGO; CKD-F, FRS; CKD-F, CKD-PC; CKD-F, KFRE; CKD-F, KDIGO; FRS, CKD-PC; FRS, KFRE; FRS, KDIGO; CKD-PC, KFRE; CKD-PC, KDIGO; KFRE, KDIGO; CKD-F, FRS, CKD-PC; CKD-F, FRS, KFRE; CKD-F, FRS, KDIGO; CKD-F, CKD-PC, KFRE; CKD-F, CKD-PC, KDIGO; CKD-F, KFRE, KDIGO; FRS, CKD-PC, KFRE; FRS, CKD-PC, KDIGO; FRS, KFRE, KDIGO; CKD-PC, KFRE, KDIGO; CKD-F, FRS, CKD-PC, KFRE; CKD-F, FRS, CKD-PC, KDIGO; CKD-F, FRS, KFRE, KDIGO; CKD-F, CKD-PC, KFRE, KDIGO; FRS, CKD-PC, KFRE, KDIGO; and CKD-F, FRS, CKD-PC, KFRE, KDIGO.

In examples, before a final prediction of CKD risk 2700 is output, an indeterminate group may be removed in stage 2600. This indeterminate group may include, for example, around 2 to 3% of patients who have healthy eyes, but poor kidney function. In examples, the cases targeted for removal may be those predicted as negative by one of the models, but predicted as positive by at least two of the other models.

The output 2700 may presented in a number of ways. In examples the output 2700 may be: an indication of the risk of developing CKD within a time period; mapped to one of the KDIGO thresholds; an eGFR threshold.

### 3.1 Retinal Predictor Models

In the illustrated example of FIG. 4A, each ensemble targets a unique label or signal in the fundus images, estimating the following variables: 1. Maculopathy grading CNNs 2218 (see, e.g. FIG. 4B)—pathological conditions of the macula, graded on a scale; 2. Retinopathy grading CNNs 2220 (see, e.g., FIG. 4C)—damage to the retina caused by diabetes, graded on a scale; 3. Retinal age CNNs 2222 (see, e.g., FIG. 4D); 4. Albumin-to-Creatinine Ratio (ACR) or Albuminuria CNNs 2210 (see, e.g., FIG. 4E)—changes related to Albuminuria (ACR); 5. eGFR CNNs 2212 (see, e.g., FIG. 4F)—changes related to eGFR; 6. Systolic Blood Pressure (SBP) CNNs 2216 (see, e.g., FIG. 4G)—changes related to systolic blood pressure elevation; 7. Total Cholesterol (T Chol) CNNs 2224 (see, e.g., FIG. 4H)—changes related to total cholesterol; 8. HbA1c CNNs 2214 (see, e.g., FIG. 4I)—changes related to HbA1c elevation; 9. Race predictor CNNs 2226 (see, e.g., FIG. 4J)—predicting the race/ethnicity of the patient; 10. Smoking predictor CNNs 2228 (see, e.g., FIG. 4K)—predicting the smoking status of the individual, and 11. CVD risk biomarker velocity predictor CNNs 2230 (see, e.g., FIG. 4L)—predicting the biomarker velocity of a CVD risk.

In an example, the CVD risk biomarker may be derived from the CLAIR deep learning (DL) model (e.g., as described in U.S. Pat. No. 11,766,223 the entire contents of which are incorporated herein by reference). CVD risk biomarker velocity predictor CNNs 2230 may be trained on longitudinal data; with the CLAIR slope calculated as (CLAIR (t2)—CLAIR (t1))/(t2−t1) and the CNN 2230 trained to predict the CLAIR slope using the retinal images at t1.

### 2.3 CKD Risk Prediction

As described above, CKD risk prediction may be based on the outputs of one or more CKD risk prediction models, including: a KIDGO model 2300, a Chronic Kidney Disease Prognosis Consortium (CKD PC) model 2302, a KFRE model 2304, Framingham Risk Score based model 2306, extended Framingham Risk Score based model 2306a, and Framingham CKD risk (CKD-F) based model 2308.

### 2.3.1 KDIGO Model

Referring to FIG. 4M, the exemplary KIDGO based model 2300 uses the following inputs: age, hypertension medication, diabetic medications, smoking status, CVD history, eGFR, ACR, SBP, R grade, M grade, and race/ethnicity.

### 2.3.2 Chronic Kidney Disease Prognosis Consortium (CKD PC) Model

Referring to FIG. 4N, the exemplary CKD PC based model 2302 uses the following inputs: age, gender, diabetes, CVD history, eGFR, ACR, SBP, and race/ethnicity.

A discussion of CKD PC risk equations may be found in “Nelson R G, Grams M E, Ballew S H, et al. Development of Risk Prediction Equations for Incident Chronic Kidney Disease. JAMA. 2019; 322 (21): 2104-2114. doi: 10.1001/jama.2019.17379”, the entire contents of which are incorporated herein by reference.

### 2.3.3 Kidney Failure Risk Equation (KFRE) Model

Referring to FIG. 4O, the KFRE based model 2304 uses the following inputs: age, gender, diabetes, CVD history, eGFR, ACR, SBP, and race/ethnicity.

A discussion of KFRE risk equations may be found in “Tangri N, Grams M E, Levey A S, et al. Multinational Assessment of Accuracy of Equations for Predicting Risk of Kidney Failure: A Meta-analysis. JAMA. 2016; 315 (2): 164-174. doi: 10.1001/jama.2015.18202”, the entire contents of which are incorporated herein by reference.

### 2.3.4 Framingham Risk Score (FRS) Model

Referring to FIG. 4P, the Framingham Risk Score based model 2306 uses the following inputs: age, gender, diabetes, smoking status, medication, eGFR, ACR, SBP, and T Chol.

In examples, an extended Framingham Risk Score based model 2306a (see, e.g., FIG. 4Q) may be used to predict a risk of developing CKD over a longer time scale.

A discussion of FRS and extended FRS equations may be found in “Lee C, Yun H R, Joo Y S, Lee S, Kim J, Nam K H, Jhee J H, Park J T, Yoo T H, Kang S W, Han S H. Framingham risk score and risk of incident chronic kidney disease: A community-based prospective cohort study. Kidney Res Clin Pract. 2019 Mar. 31; 38 (1): 49-59. doi: 10.23876/j.krcp.18.0118. PMID: 30897893; PMCID: PMC6481968”, the entire contents of which are incorporated herein by reference.

### 2.3.5 Framingham CKD Risk (CKD-F) Model

Referring to FIG. 4R, the exemplary Framingham CKD risk (CKD-F) based model 2308 uses the following inputs: age, gender, diabetes, CVD history, eGFR, ACR, SBP, and race/ethnicity.

A discussion of CKD-F risk equations may be found in “O'Seaghdha C M, Lyass A, Massaro J M, Meigs J B, Coresh J, D'Agostino R B Sr, Astor B C, Fox C S. A risk score for chronic kidney disease in the general population. Am J Med. 2012 March; 125 (3): 270-7. doi: 10.1016/j.amjmed.2011.09.009. PMID: 22340925; PMCID: PMC3285426”, the entire contents of which are incorporated herein by reference.

2.3.6 Integrating eGFR and ACR into the Framingham Risk Score

As the FRS does not have any renal markers as inputs, in examples of the present technology the FRS model 2306 may be enhanced by adding normalized eGFR and log-transformed ACR. It is envisaged that ACR may provide a stronger contribution to kidney risk prediction than eGFR.

In examples, the extended FRS model 2306a may use a squared term for eGFR (in addition to log-transformed ACR). It is believed that eGFR as a linear term may not provide a meaningful improvement to correlation with CKD measurements. It is believed this is likely due to the distribution of eGFR values in the dataset used—most participants had eGFR values clustered above 60 (i.e., in the G1-G2 range), indicating preserved kidney function. In this range, linear variations in eGFR do not correspond to significant shifts in CKD risk, so the model underestimated the contribution of lower (more clinically relevant) eGFR values. In contrast, ACR values showed a more balanced distribution across A1-A3 categories. Including a squared term for eGFR allowed the model to better capture its non-linear relationship with CKD risk.

### 4. Exemplary System and Subsystem

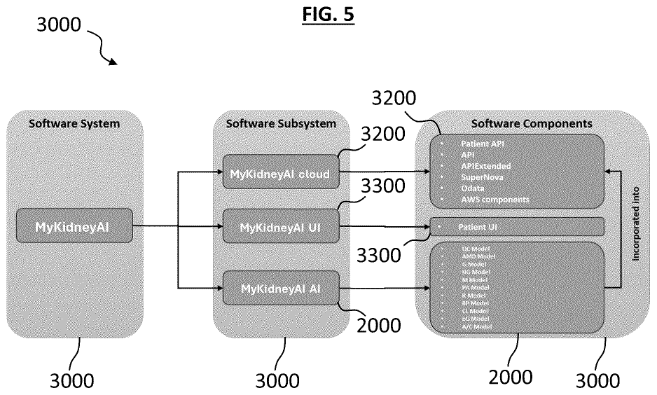

FIG. 5 illustrates an exemplary system 3000 in which examples of the present technology may be implemented. The system 3000 includes CKD AI subsystem 2000 (e.g., substantially as described with reference to FIG. 2A and/or FIG. 4A), cloud services subsystem 3200, and user interface (UI) subsystem 3300). The cloud services subsystem 3200 comprises: Patient API; API; APIExtended; SuperNova; Odata; AWS components.

### 5. Interpretation

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the field of endeavour in any country in the world.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The present disclosure may also be said broadly to consist in the parts, elements and features referred to or indicated in the specification of the application, individually or collectively, in any or all combinations of two or more of said parts, elements or features. Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the present disclosure and without diminishing its attendant advantages. It is therefore intended that such changes and modifications be included within the present disclosure as defined by the appended claims.

1. A method of determining an indication of risk of chronic kidney disease (CKD) of an individual, comprising:
determining an indication of risk of chronic kidney disease (CKD) by a deep learning model based on one or more fundus images.

2. The method of claim 1, wherein the deep learning model comprises a plurality of retinal predictor models, and the method comprises processing the one or more fundus images using the plurality of retinal predictor models and outputting at least one feature from each of the retinal predictor models.

3. The method of claim 2, wherein the plurality of retinal predictor models are configured to output at least two of the following features: Estimated Glomerular Filtration Rate (eGFR), Albumin-to-Creatinine Ratio (ACR), Systolic Blood Pressure (SBP), Retinopathy grade, and Maculopathy grade.

4. The method of claim 3, wherein the plurality of retinal predictor models are further configured to output at least one of the following features: race and/or ethnicity, Total Cholesterol, Retinal Age, Smoking status, and a cardiovascular disease (CVD) risk biomarker velocity.

5. The method of claim 2, wherein the deep learning model comprises at least one CKD risk prediction model, and the method comprises processing the features output by the plurality of retinal predictor models using the at least one CKD risk prediction model to determine the indication of risk of chronic kidney disease (CKD).

6. The method of claim 5, wherein the at least one CKD risk prediction model comprises one or more of: a KDIGO model, a Framingham CKD risk (CKD-F) model, Chronic Kidney Disease Prognosis Consortium (CKD PC) model, a Kidney Failure Risk Equation (KFRE), and a Framingham Risk Score (FRS) model.

7. The method of claim 5, wherein the at least one CKD risk prediction model comprises a plurality of CKD risk prediction models, and weighted outputs of the plurality of CKD risk prediction models contribute to determining the indication of risk of chronic kidney disease (CKD).

8. The method of claim 5, wherein the method comprises inputting at least one patient demographic into the at least one CKD risk prediction model, wherein the at least one patient demographic comprises one or more of: Age, Gender, diabetic status, race and/or ethnicity, smoking status, CVD history, and hypertension medications.

9. A system comprising:
a memory storing program instructions; and
at least one processor configured to execute program instructions stored in the memory, wherein the program instructions cause the processor to perform a method of determining an indication of risk of chronic kidney disease (CKD) of an individual, comprising:
determining an indication of risk of chronic kidney disease (CKD) by a deep learning model based on one or more fundus images.

10. The system of claim 9, wherein the deep learning model comprises a plurality of retinal predictor models, and the at least one processor is further configured to process the one or more fundus images using the plurality of retinal predictor models and output at least one feature from each of the retinal predictor models.

11. The system of claim 10, wherein the plurality of retinal predictor models are configured to output at least two of the following features: Estimated Glomerular Filtration Rate (eGFR), Albumin-to-Creatinine Ratio (ACR), Systolic Blood Pressure (SBP), Retinopathy grade, and Maculopathy grade.

12. The system of claim 11, wherein the plurality of retinal predictor models are further configured to output at least one of the following features: race and/or ethnicity, Total Cholesterol, Retinal Age, Smoking status, and a cardiovascular disease (CVD) risk biomarker velocity.

13. The system of claim 10, wherein the deep learning model comprises at least one CKD risk prediction model, and the at least one processor is further configured to process the features output by the plurality of retinal predictor models using the at least one CKD risk prediction model to determine the indication of risk of chronic kidney disease (CKD).

14. The system of claim 13, wherein the at least one CKD risk prediction model comprises one or more of: a KDIGO model, a Framingham CKD risk (CKD-F) model, Chronic Kidney Disease Prognosis Consortium (CKD PC) model, a Kidney Failure Risk Equation (KFRE), and a Framingham Risk Score (FRS) model.

15. The system of claim 13, wherein the at least one CKD risk prediction model comprises a plurality of CKD risk prediction models, and weighted outputs of the plurality of CKD risk prediction models contribute to determining the indication of risk of chronic kidney disease (CKD).

16. The system of claim 13, wherein the at least one processor is configured to input at least one patient demographic into the at least one CKD risk prediction model, wherein the at least one patient demographic comprises one or more of: Age, Gender, diabetic status, race and/or ethnicity, smoking status, CVD history, and hypertension medications.

17. A computer program product, the computer program product comprising:
a non-transitory computer-readable medium having computer-readable program code stored thereon, the computer-readable program code comprising instructions that when executed by a processor, cause the processor to perform a method of determining an indication of risk of chronic kidney disease (CKD) of an individual, comprising:
determining an indication of risk of chronic kidney disease (CKD) by a deep learning model based on one or more fundus images.
